(19) United States
(12) Patent Application Publication
(10) Pub. No.: US 2023/0153996 A1
(43) Pub. Date: May 18, 2023

(54) ULTRASOUND DEVICE AND METHOD FOR ACQUIRING PHYSIOLOGICAL PARAMETER(S) THEREBY

(71) Applicant: SHENZHEN MINDRAY BIO-MEDICAL ELECTRONICS CO., LTD., SHENZHEN (CN)

(72) Inventors: Kun XIONG, SHENZHEN (CN); Haitang CHEN, SHENZHEN (CN); Shuo LIU, SHENZHEN (CN); Tianshu LI, SHENZHEN (CN)

(21) Appl. No.: 17/984,115

(22) Filed: Nov. 9, 2022

(30) Foreign Application Priority Data
Nov. 17, 2021 (CN) 202111362428.7

(51) Int. Cl. G06T 7/00; A61B 8/08; A61B 8/00

(52) U.S. Cl. CPC G06T 7/0012; A61B 8/488; A61B 8/469; A61B 8/461; G06T 2207/10132; G06T 2207/30048; G06T 2207/30061; G06T 2207/30101; G06T 2207/30016

(57) ABSTRACT

Disclosed are an ultrasound device and method for acquiring physiological parameter(s) thereby. The method comprises: acquiring ultrasonic data of a target object, the ultrasonic data including at least an ultrasound image; performing image recognition on the ultrasound image to acquire an image recognition result; acquiring physiological parameter(s) corresponding to the image recognition result from a bedside device, the physiological parameter(s) being acquired by detecting the target object by the bedside device; and displaying the acquired physiological parameter(s) and the ultrasound image. By means of the ultrasound device and the method for acquiring physiological parameter(s) thereby according to the present disclosure, relevant physiological parameter(s) can be automatically obtained from the bedside device and displayed by the ultrasound device; and in this way, the relevant physiological parameter(s) can be quickly provided to the doctor, reducing the doctor's operations and effectively improving the efficiency of the doctor's diagnosis.

### TECHNICAL FIELD

The present disclosure relates to ultrasonography, in particular to ultrasound devices and methods for acquiring physiological parameter(s) thereby.

### BACKGROUND OF THE INVENTION

Bedside devices such as monitors can provide main parameter(s) information for medical clinical diagnosis at present. Through various functional modules, important parameter(s) such as human ECG signals, heart rate, oxygen saturation, blood pressure, respiratory rate, body temperature can be monitored in real time, so as to realize the monitoring and early warning of various parameter(s). Because of its simplicity, rapidity, accuracy, repeatability, non-invasiveness, wide application range, and no radiation, ultrasound devices have been widely used in point of care (POC) clinical scenes, including intensive care unit (ICU) and emergency department. Ultrasound devices are fast and effective means in the assessment of respiratory system, circulatory system, focused assessment with sonography for trauma (FAST), blood vessels, abdomen and other related issues.

Bedside devices (e.g. monitors) and ultrasound devices now are independent of each other, and have not been interconnected in a true sense. However, clinicians need to combine multimodal information for comprehensive analysis and decision-making, such as combining blood pressure, blood oxygen and echocardiography to determine the state of blood flow, or combining ventilator parameter(s) and ultrasonic diaphragm information to determine the respiratory state. When using an ultrasound device for diagnosing and acquiring physiological parameter(s) from a monitor associated with the ultrasound device, a doctor needs to manually screen and match many monitored parameter(s); in this regard, related operations is too cumbersome to quickly, accurately and conveniently obtain required physiological parameter(s), and it is unable to make rational use of available equipment. For example, sometimes when ultrasonic measurements on a patient require ECG waveforms and respiratory waves to perform calibration, the doctor is often reluctant to connect the ECG module, because electrodes of the monitor have been attached to the patient.

Therefore, it is necessary to provide a scheme to realize interconnection of parameter(s) among bedside equipment and ultrasound devices.

### SUMMARY OF THE INVENTION

The present disclosure is made in light of the problems above mentioned. According to an aspect of the present disclosure, a method for acquiring physiological parameter(s) by an ultrasound device is provided. The method may comprises: acquiring ultrasonic data of a target object, the ultrasonic data including at least an ultrasound image; performing image recognition on the ultrasound image to acquire an image recognition result; acquiring physiological parameter(s) corresponding to the image recognition result from a bedside device, the physiological parameter(s) being acquired by detecting the target object by the bedside device; and displaying the acquired physiological parameter(s) and the ultrasound images.

According to another aspect, there is provided a method for acquiring physiological parameter(s) by an ultrasound device. The method may comprise: acquiring ultrasonic data of a target object, the ultrasonic data including at least an ultrasound image; performing image recognition on the ultrasound image to acquire an image recognition result; acquiring physiological parameter(s) corresponding to the image recognition result from a bedside device, the physiological parameter(s) being acquired by detecting the target object by the bedside device; and displaying the acquired physiological parameter(s) and the ultrasound image.

According to yet another aspect, there is provided a method for acquiring physiological parameter(s) by an ultrasound device. The method may comprise: acquiring ultrasonic data of a target object, the ultrasonic data including ultrasonic examination mode and/or ultrasonic body mark associated with the target object; determining an examination part of the target object based on the ultrasonic examination mode and/or ultrasonic body mark; acquiring physiological parameter(s) corresponding to the examination part from a bedside device, the physiological parameter(s) being acquired by detecting the target object by the bedside device; and displaying the acquired physiological parameter(s) and the ultrasound image of the target object.

According to still yet another aspect, there is provided an ultrasound device comprising a transmitting circuit, a receiving circuit, an ultrasonic probe, a processor and a display, wherein: the transmitting circuit is configured to control the ultrasonic probe to transmit ultrasonic waves to the target object; the receiving circuit is configured to control the ultrasonic probe to receive ultrasonic echoes and obtain ultrasonic echo signals from the ultrasonic echoes; the processor is configured to perform ultrasonic imaging based on the ultrasonic echo signals; the display is configured to display the data outputted by the processor; and the processor is further configured to execute the method for acquiring the physiological parameter(s) by an ultrasound device mentioned above.

By means of the ultrasound device and the method for acquiring physiological parameter(s) thereby according to the embodiments of the present disclosure, relevant physiological parameter(s) can be automatically obtained from the bedside device and displayed by the ultrasound device; and in this way, the relevant physiological parameter(s) can be quickly provided to the doctor, reducing the doctor's operations and effectively improving the efficiency of the doctor's diagnosis.

### BRIEF DESCRIPTION OF THE DRAWINGS

The above and other purposes, features and advantages of the present disclosure will become more apparent from the detailed description of the embodiments of the present disclosure in conjunction with the accompanying drawings which are intended to provide a further understanding of the embodiments of the present disclosure. The accompanying drawings constitute a part of the specification to explain the present disclosure together with the embodiments of the present disclosure, and should not be considered as a limitation to the present disclosure. The same or similar reference numbers in the drawings generally refer to the same or similar components or steps, in which:

FIG. 1 is a schematic flowchart of a method for acquiring physiological parameter(s) by an ultrasound device according to an embodiment of the present disclosure;

FIG. 2 is an illustrative diagram of matching corresponding parameter(s) in accordance with the result of recognition performed on the ultrasound image in a method for acquiring physiological parameter(s) by an ultrasound device according to an embodiment of the present disclosure;

FIG. 3 is an illustrative diagram of matching corresponding parameter(s) in combination with multimodal information in a method for acquiring physiological parameter(s) by an ultrasound device according to an embodiment of the present disclosure;

FIG. 4 is an illustrative diagram of matching corresponding parameter(s) dependent on user input in a method for acquiring physiological parameter(s) by an ultrasound device according to an embodiment of the present disclosure;

FIG. 5 is a schematic diagram of highlighting matched parameter(s) on a bedside device in a method for acquiring physiological parameter(s) by an ultrasound device according to an embodiment of the present disclosure.

FIG. 6 is a schematic flowchart of a method for acquiring physiological parameter(s) by an ultrasound device according to another embodiment of the present disclosure;

FIG. 7 is a schematic flowchart of a method for acquiring physiological parameter(s) by an ultrasound device according to yet another embodiment of the present disclosure;

FIG. 8 is an illustrative diagram of matching corresponding parameter(s) dependent on an examination mode in a method for acquiring physiological parameter(s) by an ultrasound device according to yet another embodiment of the present disclosure; and

FIG. 9 is a schematically structural block diagram of an ultrasound apparatus according to an embodiment of the present disclosure.

### DETAILED DESCRIPTION

In order to make the objectives, technical solutions and advantages of the present disclosure more apparent, exemplary embodiments according to the present disclosure will be described in detail below with reference to the accompanying drawings. Obviously, the described embodiments are only a part of the embodiments of the present disclosure, rather than all the embodiments of the present disclosure. It should be understood that the present disclosure is not limited by the exemplary embodiments described herein. Based on the embodiments of the present disclosure described in the present disclosure, all other embodiments obtained by those skilled in the art without creative efforts shall fall within the protection scope of the present disclosure.

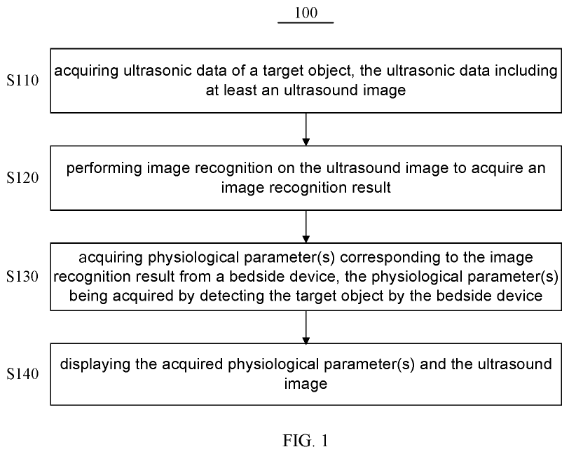

FIG. 1 shows a schematic flowchart of a method 100 for acquiring physiological parameter(s) by an ultrasound device according to an embodiment of the present disclosure. As shown in FIG. 1, the method 100 for acquiring the physiological parameter(s) by an ultrasound device may include the following steps:
step 110: acquiring ultrasonic data of a target object, the ultrasonic data including at least an ultrasound image;
step 120: performing image recognition on the ultrasound image to acquire an image recognition result;
step 130: acquiring physiological parameter(s) corresponding to the image recognition result from a bedside device, the physiological parameter(s) being acquired by detecting the target object by the bedside device; and
step 140: displaying the acquired physiological parameter(s) and the ultrasound image.

In an embodiment of the present disclosure, when a user (doctor) uses an ultrasound device to perform ultrasound imaging on a target object to assist in diagnosis, or when the user reviews ultrasound image(s) to assist in diagnosis, the ultrasound device can automatically obtain some relevant physiological parameters from a bedside device to allow the doctor to diagnose in combination with the ultrasound images and the physiological parameters from the bedside device. Specifically, the ultrasound device can acquire ultrasound image(s) of the target object, perform image recognition on the ultrasound image(s), obtain corresponding physiological parameter(s) (which may be acquired by the bedside device detecting the target object) from the bedside device according to the image recognition result, and display the acquired physiological parameter(s) and ultrasound image(s) to the user. Therefore, with the method for acquiring the physiological parameter(s) by an ultrasound device according to the embodiment of the present disclosure, relevant physiological parameter(s) from the bedside device can be automatically acquired on the ultrasound device. In this regard, such parameter(s) can be provided quickly to the doctor, reducing the doctor's operations, and effectively improving the doctor's diagnosis efficiency.

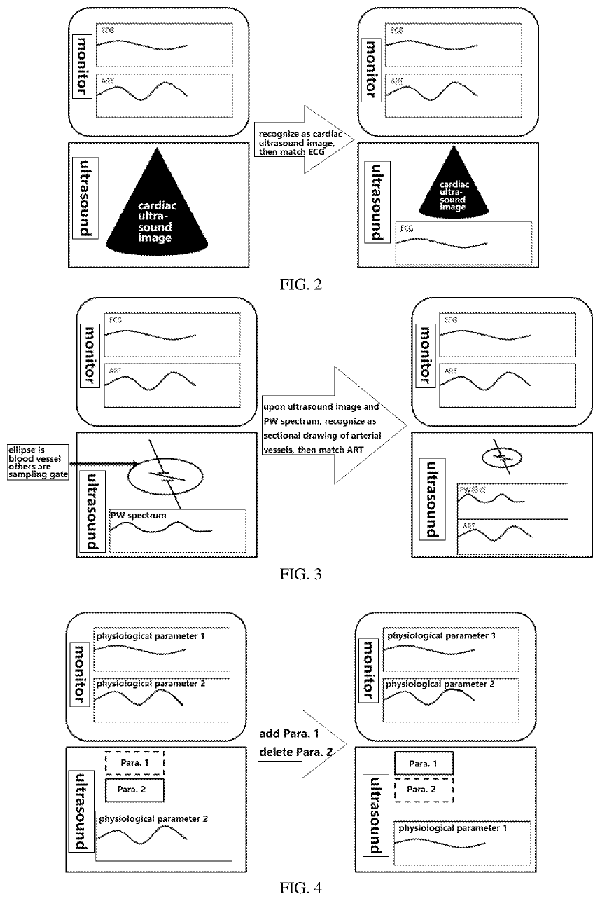

In an embodiment of the present disclosure, the ultrasound image in step 110 may include at least a grayscale image, and performing image recognition on the ultrasound image to acquire an image recognition result mentioned above may include: performing image recognition on the grayscale image to obtain the image recognition result. In this embodiment, the corresponding physiological parameter(s) can be acquired from the bedside device according to the image recognition result of the grayscale image. In an embodiment of the present disclosure, the image recognition result may be an examination part of the target object, and the physiological parameter(s) corresponding to the image recognition result may include the physiological parameter(s) corresponding to the examination part. The following is an exemplary description in conjunction with FIG. 2.

FIG. 2 shows an example of matching corresponding parameter(s) in accordance with the recognition result of the ultrasound image in the method for acquiring physiological parameter(s) by an ultrasound device according to an embodiment of the present disclosure. In the example shown in FIG. 2, the ultrasound device performs automatic image recognition on an acquired ultrasound image, and recognizes the image as a sectional drawing of the heart before determining that the current checking point is the heart. Therefore, the ultrasound device can obtain physiological parameter(s) corresponding to the heart from a bedside device (e.g. a monitor), such as electrocardiographic (ECG) waveform parameter(s), respiratory wave parameter(s) and blood oxygen parameter(s), where the ECG waveform parameter(s) can be used to evaluate information about the rhythm of cardiac motion, and the respiratory wave parameter(s) and blood oxygen parameter(s) can be used to assess overall information on circulatory function. As shown in FIG. 2, the ECG parameter(s) and arterial pressure (ART) parameter(s) are displayed on the current monitor; and after the echocardiogram is recognized, the ultrasound device can match (acquire) the ECG waveform from the monitor and display on its own display the echocardiogram together with the ECG waveform obtained from the monitor.

Here are some other examples of matching corresponding parameter(s) according to the recognition results of the ultrasound image. In one example where the bedside device is a monitor, when the image recognition result is recognized as a section of the person's brain, the physiological parameter(s) obtained from the bedside device may include at least one of blood pressure parameter(s) and blood oxygen parameter(s), by which overall information on cerebral blood flow is evaluated. In another example where the bedside device is still a monitor, when the image recognition result is recognized as a section of the person's blood vessels, the physiological parameter(s) obtained from the bedside device may include blood pressure parameter(s), ECG waveform parameter(s) and blood oxygen parameter(s), by which overall information about hemodynamics is evaluated. In yet another example where the bedside device is a monitor, when the image recognition result is recognized as a section of the person's lung(s), the physiological parameter(s) obtained from the bedside device may include at least one of ECG waveform parameter(s) and respiratory wave parameter(s), by which overall information on pulmonary function is evaluated. In yet still another example where the bedside device is a monitor, when the image recognition result is recognized as a section of the person's abdomen, the physiological parameter(s) obtained from the bedside device may include at least one of ECG waveform parameter(s), respiratory wave parameter(s), and blood oxygen parameter(s), by which information about blood flow perfusion on the viscera is evaluated. In other examples where the bedside device is a monitor, when the image recognition result is recognized as a section of other parts of the person's body, the physiological parameter(s) obtained from the bedside device can include ECG waveform parameter(s).

In this embodiments of the present disclosure, the aforesaid method for automatically recognizing the grayscale image to determine the class of the ultrasound image and the key structure contained therein may include: recognizing the classification of the ultrasound image and information on the key structure contained therein by algorithms such as classifying and target detection. It can be realized by deep learning or other machine learning and image processing methods, including (but not limited to) the ways as follows.

When using deep learning to classify images, it is first necessary to construct a database of ultrasound images which contains the class label of each image, that is, the class to which the image belongs. CNN models including AlexNet, VGG, ResNet, Inception, and MobileNet can be applied to image classification. The model is trained using the constructed database of ultrasound images. Then the images to be tested are inputted into the trained model in turn to output the probability distribution of classification of the images, and the label corresponding to the maximum probability outputted is selected as the class of each image to be tested (i.e., the class to which the image to be tested belongs can be determined).

When using deep learning for target detection, it is first necessary to construct a database of ultrasound images, in which each image is labeled with its key anatomical structure including whether a key structure exists and the coordinate information of the block closely surrounding it. Faster-RCNN, YOLO, SSD, RetinaNet, EfficientDet, FCOS, CenterNet and other models can be used for target detection. The model is trained using the constructed database of ultrasound images. Then the images to be tested are inputted into the trained model in turn to output whether each frame of image contains the key structure.

When using other machine learning algorithms to classify images, it may first perform feature extraction on the target, then match the extracted features with the database and classify with a discriminator including KNN, SVM, random forest, neutral network. The feature extraction can be implemented by traditional methods such as PCA, LDA, Harr feature and texture feature, or it may be realized by deep neural network.

When using other machine learning algorithms for target detection, it may first obtain a set of candidate rectangular regions of interest (ROIs) in respective ultrasound images by means of such as sliding window or selective search, and perform feature extraction on each candidate rectangular ROI to acquire traditional features including PCA, LDA , HOG, Harr, LBP and SIFT or features extracted by neural network. The extracted features then are matched with the extracted features of marked ROIs in the image database, and classified by linear classifier, SVM or simple neural network, etc. before determining whether the current candidate rectangular region contains key structures.

In another embodiment of the present disclosure, the ultrasound image in step 110 may include a grayscale image acquired under grayscale imaging mode and an ultrasound image under non-grayscale imaging mode; and performing image recognition on the ultrasound image may include that: performing image recognition on the combination of the gray image and the ultrasound image in non-grayscale imaging mode. In this embodiment, image recognition can be performed based on multimodal ultrasonic information to obtain more accurate image recognition results. The following will be described in connection with FIG. 3.

FIG. 3 shows an example of matching corresponding parameter(s) in combination with multimodal information in a method for acquiring physiological parameter(s) by an ultrasound device according to an embodiment of the present disclosure. In the example shown in FIG. 3, the grayscale image acquired by the ultrasound device is a grayscale image of blood vessels, and the non-grayscale imaging mode may include a pulsed Doppler (PW) imaging mode. In this respect, with the grayscale image of blood vessels and PW spectrum obtained under the PW imaging mode, the blood vessels may be identified as arterial or venous vessels by the ultrasound device. When the blood vessels are identified as arterial vessels, the physiological parameter(s) obtained by the bedside device may include arterial pressure parameter(s); and when the blood vessels are identified as venous vessels, the physiological parameter(s) obtained by the bedside device may include venous pressure parameter(s). As shown in FIG. 3, the current monitor may display the ECG parameter(s) and the arterial pressure (ART) parameter(s); and the ultrasound device may, with the grayscale image of blood vessels and PW spectrum obtained under the PW imaging mode, recognize the blood vessels as arterial vessels before matching (acquiring) the ART parameter(s) from the monitor and demonstrate on its own display the PW spectrum obtained under the PW imaging mode and the ART waveform obtained from the monitor.

In an embodiments of the present disclosure, the aforesaid method for automatically recognizing the ultrasound image with the combination of the grayscale image and other multimodal information may include: recognizing the classification of the ultrasound image and information on the key structure contained therein by algorithms such as classifying and target detection. It can be realized by deep learning or other machine learning and image processing methods, including (but not limited to) the ways as follows.

When using deep learning to classify images, it is first necessary to construct a database of ultrasound images which contains the class label of each image, that is, the class to which the image belongs. CNN models including AlexNet, VGG, ResNet, Inception, and MobileNet can be applied to image classification. The model is trained using the constructed database of ultrasound images. Then the images to be tested are inputted into the trained model in turn to output the probability distribution of classification of the images, and the label corresponding to the maximum probability outputted is selected as the class of each image to be tested (i.e. the class to which the image to be tested belongs can be determined).

When using deep learning for target detection, it is first necessary to construct a database of ultrasound images, in which each image is labeled with its key anatomical structure including whether a key structure exists and the coordinate information of the block closely surrounding it. Faster-RCNN, YOLO, SSD, RetinaNet, EfficientDet, FCOS, CenterNet and other models can be used for target detection. The model is trained using the constructed database of ultrasound images. Then the images to be tested are inputted into the trained model in turn to output whether each frame of image contains the key structure.

When using other machine learning algorithms to classify images, it may first perform feature extraction on the target, then match the extracted features with the database and classify with a discriminator including KNN, SVM, random forest, neutral network. The feature extraction can be implemented by traditional methods such as PCA, LDA, Harr feature and texture feature, or it may be realized by deep neural network.

When using other machine learning algorithms for target detection, it may first obtain a set of candidate rectangular ROIs in respective ultrasound images by means of such as sliding window or selective search, and perform feature extraction on each candidate rectangular ROI to acquire traditional features including PCA, LDA , HOG, Harr, LBP and SIFT or features extracted by neural network. The extracted features then are matched with the extracted features of marked ROIs in the image database, and classified by linear classifier, SVM or simple neural network, etc. before determining whether the current candidate rectangular region contains key structures.

In an embodiment of the present disclosure, the method 100 may further include: obtaining a user input which may include a physiological parameter to be added or deleted by the user; when the user input indicates adding a physiological parameter, acquiring and displaying the physiological parameter(s) to be added indicated by the user input; and when the user input indicates the deletion of a physiological parameter, deleting the physiological parameter to be deleted indicated by the user input from the displayed physiological parameter(s). The following is an exemplary description in conjunction with FIG. 4.

FIG. 4 shows an example of matching corresponding parameter(s) dependent on user input in a method for acquiring physiological parameter(s) by an ultrasound device according to an embodiment of the present disclosure. As shown in FIG. 4, the parameter(s) currently displayed on the monitor include(s) a physiological parameter 1 and a physiological parameter 2, and the physiological parameter 2 is acquired from the monitor and displayed by the ultrasound device. Subsequently, after receiving a user input indicating adding the parameter 1 and deleting parameter 2, the ultrasound device may thus no longer show the physiological parameter 2 but the physiological parameter 1 acquired from the monitor.

In an embodiment of the present disclosure, when the target object is coupled to more than one bedside device, the ultrasound device may also determine, based on the recognition result of the ultrasonic data, the bedside device(s) from which the physiological parameter(s) is(are) to be acquired, so as to acquire the corresponding physiological parameter(s) from the determined bedside device(s). For example, when the recognition result corresponds to the lung(s), it may be necessary to acquire the physiological parameter(s) from a ventilator and also from the monitor; in this respect, the ventilator and the monitor may be selected from the bedside devices coupled to the target object to acquire the physiological parameter(s) from thereto.

In an embodiment of the present disclosure, when the ultrasound device acquires the physiological parameter(s) from the bedside device, it needs to establish a network connection with the bedside device. For example, the network connection can be established by any of the following ways: the ultrasound device and the bedside device being connected to the same wireless network hotspot; the ultrasound device providing a wireless network hotspot to which the bedside device connects; the bedside device providing a wireless network hotspot to which the ultrasound device connects; and the ultrasound device and the bedside device being connected via a near-field communication network, a mobile network or a wired network.

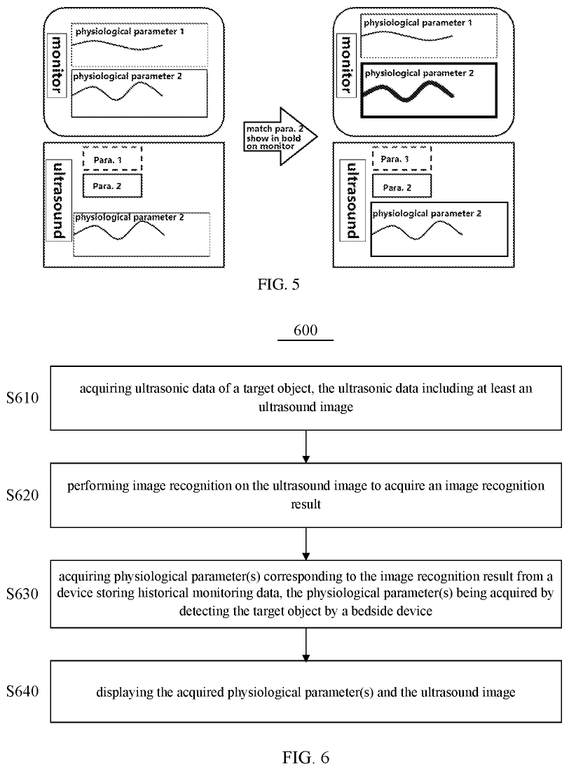

In an embodiment of the present disclosure, the physiological parameter(s) acquired from the bedside device and displayed by the ultrasound device can be highlighted shown on the bedside device at the same time, which enables the user to more clearly see the physiological parameter(s) acquired by the ultrasound device on the monitor, emphasizing the current importance of the physiological parameter(s). The following is an exemplary description in conjunction with FIG. 5.

FIG. 5 shows a schematic diagram of highlighting matched parameter(s) on a bedside device in a method for acquiring physiological parameter(s) by an ultrasound device according to an embodiment of the present disclosure. As shown in FIG. 5, the parameter(s) currently displayed on the monitor include(s) a physiological parameter 1 and a physiological parameter 2, and the parameter acquired from the monitor and displayed by the ultrasound device is physiological parameter 2; accordingly, the physiological parameter 2 on the monitor is displayed in bold, so that the doctor or an assistant can quickly observe the matched parameter(s) on the monitor.

In an embodiments of the present disclosure, the bedside device described above may include at least one of the following: a monitor, a ventilator, an anesthesia machine, and a personal computer.

With the above description, by means of the method for acquiring physiological parameter(s) by an ultrasound device according to the embodiment(s) of the present disclosure, relevant physiological parameter(s) can be automatically obtained from the bedside device and displayed by the ultrasound device based on the recognition result of the ultrasound image. In this way, the relevant physiological parameter(s) can be quickly provided to the doctor, reducing the doctor's operations and effectively improving the efficiency of the doctor's diagnosis.

The following describes a method 600 for acquiring physiological parameter(s) by an ultrasound device according to another embodiment of the present disclosure with reference to FIG. 6. As shown in FIG. 6, the method 600 for acquiring the physiological parameter(s) by the ultrasound device may include the following steps:
step 610: acquiring ultrasonic data of a target object, the ultrasonic data including at least an ultrasound image;
step 620, performing image recognition on the ultrasound image to acquire an image recognition result;
step 630, acquiring physiological parameter(s) corresponding to the image recognition result from a device storing historical monitoring data, the physiological parameter(s) being acquired by detecting the target object by a bedside device; and
step 640, displaying the acquired physiological parameter(s) and the ultrasound image.

The method 600 for acquiring physiological parameter(s) by an ultrasonic device according to an embodiment of the application is substantially similar to the method 100 for acquiring physiological parameter(s) by an ultrasonic device described above; and the difference therebetween is that, the ultrasound device may acquire corresponding physiological parameter(s) not from the bedside device, but from a device that stores historical monitoring data (such as a central station) after obtaining the image recognition result. This can be applied to non-real time applications. Due to the limited time for the bedside device to save data in non-real time application scenarios, the relevant data may not be included in the bedside device when the ultrasonic device wants to obtain data; to solve this, the ultrasound device can acquire desired physiological parameter(s) from the device storing historical monitoring data (e.g. a central station) and display it on the ultrasound device to meet the non-real-time application scenarios.

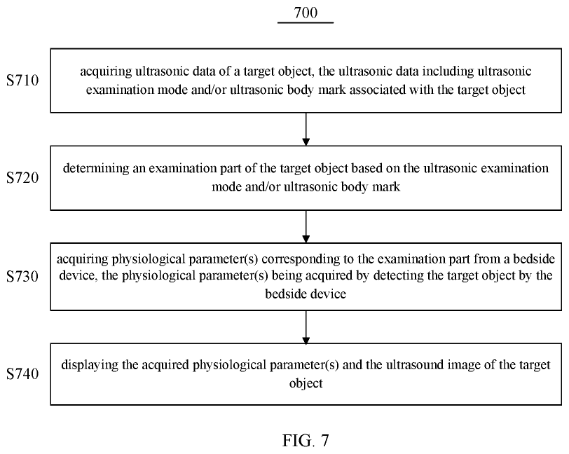

The following describes a method 700 for acquiring physiological parameter(s) by an ultrasound device according to yet another embodiment of the present disclosure with reference to FIG. 7. As shown in FIG. 7, the method 700 for acquiring the physiological parameter(s) by the ultrasound device may include the following steps:
step 710: acquiring ultrasonic data of a target object, the ultrasonic data including ultrasonic examination mode and/or ultrasonic body mark associated with the target object;
step 720, determining an examination part of the target object based on the ultrasonic examination mode and/or ultrasonic body mark;
step 730, acquiring physiological parameter(s) corresponding to the examination part from a bedside device, the physiological parameter(s) being acquired by detecting the target object by the bedside device; and
step 740, displaying the acquired physiological parameter(s) and the ultrasound image of the target object.

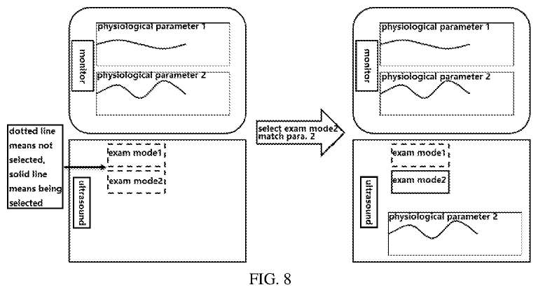

The method 700 for acquiring physiological parameter(s) by an ultrasonic device according to an embodiment of the application is substantially similar to the method 100 for acquiring physiological parameter(s) by an ultrasonic device described above; and the difference therebetween is that, the method 100 for acquiring physiological parameter(s) by an ultrasound device is about acquiring corresponding physiological parameter(s) from a bedside device based on the result of ultrasound image recognition and display it(them), while the method 700 for acquiring physiological parameter(s) by an ultrasound device is about determining an examination part of a target object based on the ultrasonic examination mode and/or the ultrasonic body mark so as to acquire corresponding physiological parameter(s) from a bedside device and display it(them). The ultrasonic examination mode may be the one currently selected by the user, such as a cardiac examination mode, a transcranial examination mode, etc. . . The ultrasonic body mark may be the one generated according to a measurement item or the one selected by the user; for example, when the current measurement item is related to the heart, a heart body mark is generated, or a heart body mark may be selected by the user. The following is an exemplary description in conjunction with FIG. 8.

FIG. 8 shows an example of matching corresponding parameter(s) dependent on examination mode in a method for acquiring physiological parameter(s) by an ultrasound device according to another embodiment of the present disclosure. As shown in FIG. 8, the parameter(s) currently displayed on the monitor include(s) a physiological parameter 1 and a physiological parameter 2, and an examination mode 1 and an examination mode 2 are included in the ultrasound device; in this respect, when a user chooses the examination mode 2, the physiological parameter 2 corresponding to the examination 2 can be acquired from the monitor and displayed by the ultrasound device.

In a further embodiment of the present disclosure, the ultrasonic data acquired in step 710 may also include an imaging mode associated with the target object, and the step 720 of determining an examination part of the target object based on the ultrasonic examination mode and/or ultrasonic body mark may include: determining the examination part of the target object based on the imaging mode in combination with the ultrasonic examination mode and/or the ultrasonic body mark. In this embodiment, the examination part of the target object is determined not only based on the ultrasonic examination mode and/or the ultrasonic body mark, but also based on the imaging mode; in this respect, it may be more accurate to determine a more precise examination part of the target object, which is beneficial to determine what more valuable physiological parameter(s) to be obtained from the bedside device.

Some examples are listed below. In one example where the bedside device is a monitor, when the ultrasonic examination mode is the cardiac examination mode and/or the ultrasonic body mark is a cardiac mark, the physiological parameter(s) acquired from the bedside device include at least one of the ECG waveform parameter(s) used for evaluating information about the rhythm of cardiac motion, the respiratory wave parameter(s) and the blood oxygen parameter(s) both used for assessing overall information on circulatory function. In another example where the bedside device is a monitor, when the ultrasonic examination mode is the transcranial examination mode and/or the ultrasonic body mark is a cerebral mark, the physiological parameter(s) acquired from the bedside device include at least one of the blood pressure parameter(s) and the blood oxygen parameter(s) for evaluating the overall information of cerebral blood flow. In yet another example where the bedside device is a monitor, and when the ultrasonic examination mode is a vascular examination mode and/or the ultrasonic body mark is a vascular mark, the physiological parameter(s) acquired from the bedside device may include at least one of the blood pressure parameter(s), the ECG waveform parameter(s), and the blood pressure parameter(s) for evaluating overall information about hemodynamics. In still yet another example where the bedside device is a monitor, when the ultrasonic examination mode is a pulmonary examination mode and/or the ultrasonic body mark is a pulmonary mark, the physiological parameter(s) acquired from the bedside device include at least one of the ECG waveform parameter(s) and the respiratory wave parameter(s) for evaluating overall information on pulmonary function. In yet still another example where the bedside device is a monitor, when the ultrasonic examination mode is an abdominal examination mode and/or the ultrasonic body mark is an abdominal mark, the physiological parameter(s) acquired from the bedside device include at least one of the ECG waveform parameter(s), the respiratory wave parameter(s) and the blood oxygen parameter(s) for evaluating information about blood flow perfusion on the viscera.

In addition, similar to the method 100 described above, the method 700 may further include: obtaining a user input which includes a physiological parameter to be added or deleted by the user; when the user input instructs adding a physiological parameter, acquiring and displaying the physiological parameter(s) to be added indicated by the user input; and when the user input indicates the deletion of a physiological parameter, deleting the physiological parameter to be deleted indicated by the user input from the displayed physiological parameter(s).

In addition, similar to the method 100 described above, in the method 700, when the target object is coupled to more than one bedside device, the ultrasound device may also determine, based on the ultrasonic data obtained in step 710, the bedside device(s) from which the physiological parameter(s) is(are) to be acquired, and acquire corresponding physiological parameter(s) from the determined bedside device(s) after establishing a network connection with the determined bedside device(s).

In addition, similar to the method 100 described above, in the method 700, the ultrasound device needs to establish a network connection with the bedside device when it acquires physiological parameter(s) from the bedside device. For example, the network connection can be established by any of the following ways: the ultrasound device and the bedside device being connected to the same wireless network hotspot; the ultrasound device providing a wireless network hotspot to which the bedside device connects; the bedside device providing a wireless network hotspot to which the ultrasound device connects; and the ultrasound device and the bedside device being connected via a near-field communication network, a mobile network or a wired network.

In addition, similar to the method 100 described above, in the method 700, the physiological parameter(s) acquired from the bedside device and displayed by the ultrasound device can be highlighted shown on the bedside device at the same time, which enables the user to more clearly see the physiological parameter(s) acquired by the ultrasound device on the monitor, emphasizing the current importance of the physiological parameter(s).

In addition, similar to the method 100 described above, in the method 700, the bedside device(s) described above may include at least one of the following: a monitor, a ventilator, an anesthesia machine, and a personal computer.

With the above description, by means of the method for acquiring physiological parameter(s) by an ultrasound device according to the embodiment(s) of the present disclosure, the examination part of the target object can be determined based on the ultrasonic examination mode and/or ultrasonic body mark and the physiological parameter(s) related to the examination part can be automatically acquired from the bedside device and displayed on the ultrasound device. In this way, the relevant physiological parameter(s) can be quickly provided to the doctor, reducing the doctor's operations and effectively improving the efficiency of the doctor's diagnosis.

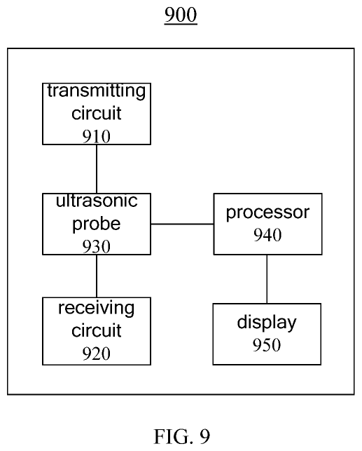

The method for acquiring physiological parameter(s) by the ultrasonic device according to an embodiment of the present disclosure is described above by way of example. An ultrasonic device provided according to another aspect of the present disclosure will be described below with reference to FIG. 9.

FIG. 9 schematically shows a structural block diagram of an ultrasound device 900 according to an embodiment of the present disclosure. As shown in FIG. 9, the ultrasound device 900 includes a transmitting circuit 910 configured to control an ultrasonic probe 930 to transmit ultrasonic waves to the target object, a receiving circuit 920 configured to control the ultrasonic probe 930 to receive ultrasonic echoes and obtain ultrasonic echo signals from the ultrasonic echoes, the ultrasonic probe 930, a processor 940 configured to perform ultrasonic imaging based on the ultrasonic echo signals, and a display 950 configured to display the data outputted by the processor 940. The processor 940 is further configured to execute the method for acquiring the physiological parameter(s) by the ultrasound device described above. Those skilled in the art can understand the structure and operation of the ultrasound device 900 in combination with the foregoing. For simplicity, it will not be repeated here.

With the above description, by means of the method for acquiring physiological parameter(s) by an ultrasound device, as well as the ultrasound device, according to the embodiment of the present disclosure, relevant physiological parameter(s) can be automatically obtained from the bedside device(s) and displayed by the ultrasound device; and in this way, the relevant physiological parameter(s) can be quickly provided to the doctor, reducing the doctor's operations and effectively improving the efficiency of the doctor's diagnosis.

While exemplary embodiments have been described herein with reference to the accompanying drawings, it should be understood that the above example embodiments are merely illustrative and are not intended to limit the scope of the disclosure thereto. Those skilled in the art may make various changes and modifications therein without departing from the scope and spirit of the disclosure. All such changes and modifications are intended to be included in the scope of the disclosure as claimed in the appended claims.

A person of ordinary skill in the art may be aware that, in combination with the examples described in the embodiments disclosed in this specification, units and algorithm steps may be implemented by using electronic hardware or a combination of computer software and electronic hardware. Whether the functions are performed by hardware or software depends on particular applications and design constraint conditions of the technical solutions. Those skilled in the art could use different methods to implement the described functions for each particular application, but such implementation should not be considered to be beyond the scope of the disclosure.

In several embodiments provided in the present disclosure, it should be understood that the disclosed devices and methods may be implemented in other ways. For example, the device embodiments described above are merely exemplary. For example, the division of units is merely a logical function division. In actual implementations, there may be other division methods. For example, a plurality of units or components may be combined or integrated into another device, or some features may be omitted or not implemented.

A large number of specific details are explained in this specification provided herein. However, it can be understood that the embodiments of the disclosure can be practiced without these specific details. In some instances, well-known methods, structures, and technologies are not shown in detail, so as not to obscure the understanding of this description.

Similarly, it should be understood that in order to simplify the disclosure and help to understand one or more of various aspects of the disclosure, in the description of the exemplary embodiments of the disclosure, various features of the disclosure are sometimes together grouped into an individual embodiment, figure or description thereof. However, the method of the disclosure should not be construed as reflecting the following intention, namely, the disclosure set forth requires more features than those explicitly stated in each claim. More precisely, as reflected by the corresponding claims, the inventive point thereof lies in that features that are fewer than all the features of an individual embodiment disclosed may be used to solve the corresponding technical problem. Therefore, the claims in accordance with the particular embodiments are thereby explicitly incorporated into the particular embodiments, wherein each claim itself serves as an individual embodiment of the disclosure.

Those skilled in the art should understand that, in addition to the case where features are mutually exclusive, any combination may be used to combine all the features disclosed in this specification (along with the appended claims, abstract, and drawings) and all the processes or units of any of methods or devices as disclosed. Unless explicitly stated otherwise, each feature disclosed in this specification (along with the appended claims, abstract, and drawings) may be replaced by an alternative feature that provides the same, equivalent, or similar object.

Furthermore, those skilled in the art should understand that although some of the embodiments described herein comprise some but not other features included in other embodiments, combinations of features of different embodiments are meant to be within the scope of the disclosure, and form different embodiments. For example, in the claims, any one of the embodiments set forth thereby can be used in any combination.

Various embodiments regarding components in the disclosure may be implemented in hardware, or implemented by software modules running on one or more processors, or implemented in a combination thereof. It should be understood for those skilled in the art that a microprocessor or a digital signal processor (DSP) may be used in practice to implement some or all of the functions of some modules according to the embodiments of the disclosure. The disclosure may further be implemented as an apparatus program (e.g. a computer program and a computer program product) for executing some or all of the methods described herein. Such a program for implementing the disclosure may be stored on a computer-readable medium, or may be in the form of one or more signals. Such a signal may be downloaded from an Internet website, or provided on a carrier signal, or provided in any other form.

It should be noted that the description of the disclosure made in the above-mentioned embodiments is not to limit the disclosure, and those skilled in the art may design alternative embodiments without departing from the scope of the appended claims. In the claims, any reference signs placed between parentheses should not be construed as limitation on the claims. The word “comprising” does not exclude the presence of elements or steps not listed in a claim. The word “a” or “an” preceding an element does not exclude the presence of a plurality of such elements. The disclosure may be implemented by means of hardware comprising several different elements and by means of an appropriately programmed computer. In unit claims listing several ultrasound devices, several of these ultrasound devices may be specifically embodied by one and the same item of hardware. The use of the terms “first”, “second”, “third”, etc. does not indicate any order. These terms may be interpreted as names.

The above is only the specific embodiment of the present disclosure or the description of the specific embodiment, and the protection scope of the present disclosure is not limited thereto. Any changes or substitutions should be included within the protection scope of the present disclosure. The protection scope of the present disclosure shall be subject to the protection scope of the claims.

1. A method for acquiring physiological parameter(s) by an ultrasound device, comprising:
acquiring ultrasonic data of a target object, the ultrasonic data including at least an ultrasound image;
performing image recognition on the ultrasound image to acquire an image recognition result;
acquiring physiological parameter(s) corresponding to the image recognition result from a bedside device or a device storing historical monitoring data, the physiological parameter(s) being acquired by detecting the target object by the bedside device; and
displaying the acquired physiological parameter(s) and the ultrasound image.

2. The method according to claim 1, wherein when the ultrasound image at least comprises a grayscale image, performing image recognition on the ultrasound image to acquire an image recognition result comprises:
performing image recognition on the grayscale image to obtain the image recognition result;
When the ultrasound image includes a grayscale image acquired under grayscale imaging mode and an ultrasound image under non-grayscale imaging mode, and
performing image recognition on the ultrasound image comprises: performing image recognition on a combination of the grayscale image and the ultrasound image under non-grayscale imaging mode.

3. (canceled)

4. The method according to claim 1, wherein the image recognition result includes an examination part of the target object, and the physiological parameter(s) corresponding to the image recognition result include(s): physiological parameter(s) corresponding to the examination part.

5. The method according to claim 4, wherein
the bedside device includes a monitor, and
when the image recognition result is recognized as a section of a heart or a section of the abdomen, the physiological parameter(s) acquired by the bedside device comprise(s) at least one of electrocardiographic waveform parameter(s), respiratory wave parameter(s) and blood oxygen parameter(s).

6. The method according to claim 4, wherein
the bedside device includes a monitor, and
when the image recognition result is recognized as a section of a brain, the physiological parameter(s) acquired by the bedside device comprise(s) at least one of pressure parameter(s) and blood oxygen parameter(s).

7. The method according to claim 4, wherein
the bedside device includes a monitor, and
when the image recognition result is recognized as a section of blood vessels, the physiological parameter(s) acquired by the bedside device comprise(s) at least one of blood pressure parameter(s), electrocardiographic waveform parameter(s) and blood oxygen parameter(s).

8. The method according to claim 4, wherein
the bedside device includes a monitor, and
when the image recognition result is recognized as a section of lung(s), the physiological parameter(s) acquired by the bedside device comprise(s) at least one of electrocardiographic waveform parameter(s) and respiratory wave parameter(s).

9. (canceled)

10. The method according to claim 3,
wherein the grayscale image includes a grayscale image of blood vessels, and the non-grayscale imaging mode includes a pulsed Doppler imaging mode; and
wherein performing image recognition on the combination of the grayscale image and the ultrasound image in non-grayscale imaging mode comprises:
identifying the blood vessels as arterial or venous vessels on the combination of the grayscale image of the blood vessels and a pulsed Doppler spectrum obtained under the pulsed Doppler imaging mode;
when the blood vessels are identified as arterial vessels, the physiological parameter(s) obtained by the bedside device include(s) arterial pressure parameter(s); and
when the blood vessels are identified as venous vessels, the physiological parameter(s) obtained by the bedside device include(s) venous pressure parameter(s).

11. The method according to claim 1, further comprising:
obtaining a user input including a physiological parameter to be added or deleted;
when the user input indicates adding a physiological parameter, acquiring and displaying the physiological parameter to be added indicated by the user input; and
when the user input indicates deleting a physiological parameter, deleting the physiological parameter to be deleted indicated by the user input from the displayed physiological parameter(s).

12.-13. (canceled)

14. The method according to claim 1, wherein the physiological parameter(s) acquired from the bedside device and displayed is(are) simultaneously highlighted shown on the bedside device.

15.-16. (Canceled)

17. A method for acquiring physiological parameter(s) by an ultrasound device, comprising:
acquiring ultrasonic data of a target object, the ultrasonic data including an ultrasonic examination mode and/or an ultrasonic body mark associated with the target object;
determining an examination part of the target object based on the ultrasonic examination mode and/or the ultrasonic body mark;
acquiring physiological parameter(s) corresponding to the examination part from a bedside device or a device storing historical monitoring data, the physiological parameter(s) being acquired by detecting the target object by the bedside device; and
displaying the acquired physiological parameter(s) and the ultrasound image of the target object.

18. The method according to claim 17, wherein the ultrasonic body mark is generated according to a measurement item or is selected by a user.

19. The method according to claim 17, wherein
the ultrasonic data further comprises an imaging mode associated with the target object, and p1 determining an examination part of the target object based on the ultrasonic examination mode and/or the ultrasonic body mark comprises:
determining the examination part of the target object based on the imaging mode in combination with the ultrasonic examination mode and/or the ultrasonic body mark.

20. The method according to claim 17, wherein
the bedside device includes a monitor, and
when the ultrasonic examination mode is a cardiac examination mode or an abdominal examination mode, and/or the ultrasonic body mark is a cardiac mark, the physiological parameter(s) acquired from the bedside device include(s) at least one of electrocardiographic waveform parameter(s), respiratory wave parameter(s), and blood oxygen parameter(s).

21. The method according to claims 17, wherein
the bedside device includes a monitor, and
when the ultrasonic examination mode is a transcranial examination mode and/or the ultrasonic body mark is a cerebral mark, or when the ultrasonic examination mode is an abdominal examination mode and/or the ultrasonic body mark is an abdominal mark, the physiological parameter(s) acquired from the bedside device include(s) at least one of blood pressure parameter(s) and blood oxygen parameter(s).

22. The method according to claims 17, wherein
the bedside device includes a monitor, and
when the ultrasonic examination mode is a vascular examination mode and/or the ultrasonic body mark is a vascular mark, the physiological parameter(s) acquired from the bedside device include(s) at least one of blood pressure parameter(s), electrocardiographic waveform parameter(s), and blood oxygen parameter(s).

23. The method according to claim 17, wherein
the bedside device includes a monitor, and
when the ultrasonic examination mode is a pulmonary examination mode and/or the ultrasonic body mark is a pulmonary mark, the physiological parameter(s) acquired from the bedside device include(s) at least one of electrocardiographic waveform parameter(s) and respiratory wave parameter(s) for evaluating overall information on pulmonary function.

24. (canceled)

25. The method according to claim 17, further comprising:
obtaining a user input including a physiological parameter to be added or deleted;
when the user input indicates adding a physiological parameter, acquiring and displaying the physiological parameter to be added indicated by the user input; and
when the user input indicates deleting a physiological parameter, deleting the physiological parameter to be deleted indicated by the user input from the displayed physiological parameter(s).

26. (canceled)

27. The method according to claim 17, wherein the physiological parameter(s) acquired from the bedside device and displayed is(are) simultaneously shown in bold or highlighted with a rectangular frame on the bedside device.

28. (canceled)

29. An ultrasound device, comprising a transmitting circuit, a receiving circuit, an ultrasonic probe, a processor and a display, wherein:
the transmitting circuit is configured to control the ultrasonic probe to transmit ultrasonic waves to the target object;
the receiving circuit is configured to control the ultrasonic probe to receive ultrasonic echoes and obtain ultrasonic echo signals from the ultrasonic echoes;
the processor is configured to perform ultrasonic imaging based on the ultrasonic echo signals; and
the processor is further configured to:
perform image recognition on the ultrasound image to acquire an image recognition result;
acquire physiological parameter(s) corresponding to the image recognition result from a bedside device or a device storing historical monitoring data, the physiological parameter(s) being acquired by detecting the target object by the bedside device; and
the display is configured to display the acquired physiological parameter(s) and the ultrasound image.
